(19) United States
(12) Patent Application Publication
(10) Pub. No.: US 2025/0108210 A1
(43) Pub. Date: Apr. 3, 2025

(54) THERMAL MAPPING FOR TREATMENT PLANNING FOR TUMOR TREATING FIELDS

(71) Applicant: Novocure GmbH, Root D4 (CH)

(72) Inventors: Nadav SHAPIRA, Haifa (IL); Yoram WASSERMAN, Haifa (IL)

(73) Assignee: Novocure GmbH, Root D4 (CH)

(21) Appl. No.: 18/885,275

(22) Filed: Sep. 13, 2024

Related U.S. Application Data
(60) Provisional application No. 63/541,435, filed on Sep. 29, 2023.

(51) Int. Cl. A61N 1/36; G16H 20/40

(52) U.S. Cl. CPC A61N 1/36002; A61N 1/36034; G16H 20/40

(57) ABSTRACT

A computer-implemented method for generating spatial thermal mapping of a subject is disclosed. The method includes: providing first signals to heat a plurality of heating elements on a garment worn by the subject; receiving second signals from a plurality of thermal sensing elements on the garment worn by the subject; ceasing the first signals; determining thermal-related values based on the second signals; and generating a spatial thermal mapping of the subject based on at least one of the determined temperatures or the determined times.

### CROSS-REFERENCE TO RELATED APPLICATIONS

This application claims priority to U.S. Provisional Application No. 63/541,435, filed Sep. 29, 2023, which is incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 illustrates a front view of a spatial thermal mapping apparatus of at least some exemplary embodiments of the present disclosure.

FIG. 2 illustrates a detailed view of a sensor assembly of a spatial thermal mapping apparatus of at least some exemplary embodiments of the present disclosure.

FIG. 3 illustrates an exemplary method for generating a spatial thermal mapping of a subject.

FIG. 4 illustrates an example display of a three-dimensional representation of a subject.

FIG. 5 illustrates an example display of a subject based on a spatial thermal mapping of the subject.

FIG. 6 illustrates an exemplary method for generating transducer layouts for applying TTFields to a subject based on a spatial thermal mapping of the subject.

FIG. 7 illustrates a schematic view of an exemplary apparatus for applying alternating electric fields.

FIG. 8A illustrates a schematic view of an exemplary design of a transducer for applying alternating electric fields.

FIG. 8B illustrates a schematic view of an exemplary design of a transducer for applying alternating electric fields.

FIG. 9 illustrates a top view of an exemplary transducer array.

FIG. 10 illustrates an exemplary computer apparatus of at least some exemplary embodiments of the present disclosure.

### DESCRIPTION OF EMBODIMENTS

As discovered by the inventors, a spatial thermal mapping of a subject may be useful in development treatment planning for applying TTFields. A spatial thermal mapping of a subject may indicate the ability of certain areas of the subject's skin to dissipate heat to cool the subject's skin. Using such a spatial thermal mapping, locations on the subject to place transducers and/or locations on the subject to avoid placing transducers may be identified and then used in treatment planning for applying TTFields. When such locations are used for placement of transducers and/or avoiding placement of transducers, an effectiveness of TTFields treatment may be increased. For example, the more heat a given area of the body can dissipate, the more TTFields can be delivered at that given area. Because a byproduct of TTFields therapy is heat generation on the surface of the skin, identifying specific points or areas on a subject that can dissipate heat better than others may be helpful. That is, transducers that generate heat may be placed at locations of subject's skin more effective at dissipating heat as identified in the spatial thermal mapping, which may result in a transducer layout that increases the effectiveness of that subject's TTFields therapy.

Despite providing benefits as discussed above, spatial thermal mapping may be problematic. Thermal imaging typically involves expensive thermal cameras that are difficult to operate and move and further involve specific imaging conditions (e.g., ambient temperature and/or temperature differential between a subject and the environment around the subject). Accordingly, thermal imaging with such systems is often difficult, cumbersome, and expensive to conduct. To address these problems, the inventors discovered an apparatus to obtain the benefits of a spatial thermal mapping of a subject (e.g., as discussed above) without involving complex, cumbersome, and expensive thermal cameras. The inventors have thereby discovered a technique for obtaining the advantages of spatial thermal mapping while avoiding the problems associated with such thermal cameras.

In some exemplary embodiments, the inventive techniques may be used to generate a spatial thermal mapping (e.g., a spatial cooling mapping) of a subject. For example, the subject may wear a snug-fitting garment (e.g., a vest or any other suitable garment for example as described herein) with a number of heating elements and thermal sensing elements. The spatial thermal mapping may be generated based on measurements of the thermal sensing elements. The spatial thermal mapping may then be used in treatment planning for TTFields to identify areas of the subject that can easily (e.g., or not easily) dissipate heat, and these identified areas can then be used to assist in identifying locations on the subject to place transducer arrays for the application of TTFields to the subject.

In some exemplary embodiments, the inventive techniques may be efficient, effective, and cost-effective techniques for performing spatial thermal mapping and for using the spatial thermal mapping in treatment planning for TTFields. The inventive techniques may be used to perform spatial thermal mapping of subjects in a wide variety of locations without the use of complex, cumbersome, and expensive equipment.

In some embodiments, the inventive techniques described herein may provide a practical application to determine a spatial thermal mapping of a subject and display a representation thereof. Signals indicative of temperature and/or time (e.g., time to cool down) may be provided from sensor assemblies of a garment worn by a subject. Data associated with signals received from such sensor assemblies may be processed to determine the spatial thermal mapping, including temperature and time data indicative of the ability of the subject's skin to cool. Transducer placement for TTFields treatment may be based on the spatial thermal map so as to determine a desired treatment plan of TTFields for the subject.

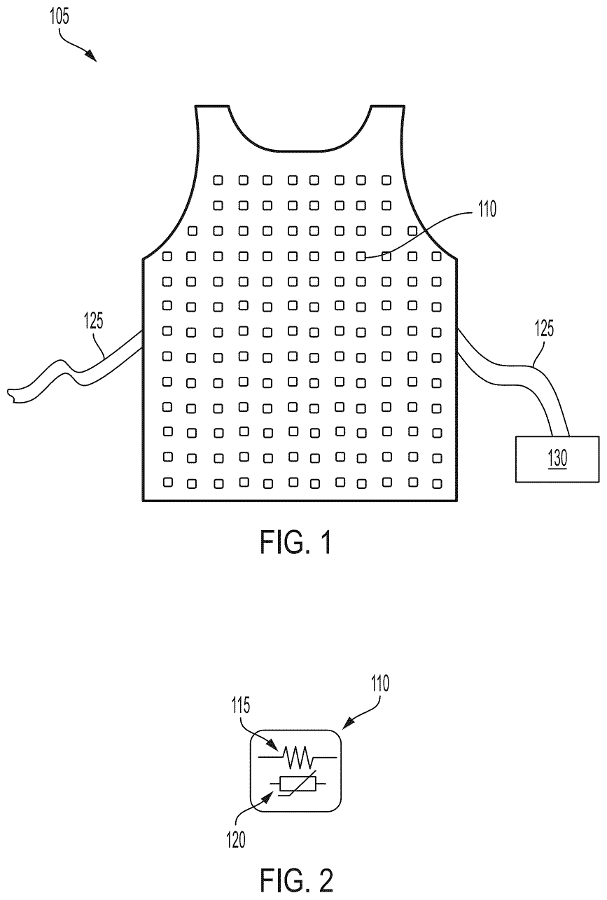

FIG. 1 illustrates a front view of a spatial thermal mapping apparatus of at least some exemplary embodiments of the present disclosure. The exemplary spatial thermal mapping apparatus may be a garment 105. Garment 105 may be a wearable garment such as, for example, a snug-fitting garment. A snug-fitting garment may be, for example, a garment that conforms to a shape of the body of the subject. For example, garment 105 may be a vest, a shirt, a helmet, a skull cap, a leg garment (e.g., a thigh garment or leggings), pants, and/or shorts.

Garment 105 may include a plurality of sensor assemblies 110. Sensor assemblies 110 may be any suitable assembly for measuring a skin temperature of a subject. Sensor assemblies 110 may be attached to garment 105 via any suitable technique (e.g., stitching, adhesive, fasteners, and/or any other suitable attachment technique). Any suitable number of sensor assemblies 110 may be attached to garment 105 such as, for example, between about 5 and about 100 sensor assemblies 110.

FIG. 2 illustrates a detailed view of a sensor assembly of a spatial thermal mapping apparatus of at least some exemplary embodiments of the present disclosure. For example, sensor assembly 110 may include a heating element 115 and a thermal sensing element 120. The garment 105 from FIG. 1 may have a same number of heating elements 115 and thermal sensing elements 120, or have a different number of heating elements 115 and thermal sensing elements 120. Heating element 115 may be any suitable heat generating element such as, for example, a resistor. Heating element 115 may be, for example, a carbon resistor, a thin or thick film resistor, a fixed or variable resistor, a wire wound resistor, a metal film resistor, or any other suitable type of resistor. Thermal sensing element 120 may be any suitable device to measure temperature such as, for example, a thermistor (e.g., a resistor having a resistance that is temperature-dependent). For example, thermal sensing element 120 may be a negative temperature coefficient thermistor or a positive temperature coefficient thermistor.

Heating element 115 and thermal sensing element 120 at each sensor assembly 110 may be separate components that are co-located together. Co-located heating element 115 and thermal sensing element 120 may be any suitable distance from each other such as, for example, in contact, immediately adjacent, or up to about 0.5 cm from each other (for example, 0.1 cm, 0.2 cm, 0.3 cm, 0.4 cm, and 0.5 cm). Heating element 115 and thermal sensing element 120 may also be integrated together as a single component. Heating element 115 of a given sensor assembly 110 may operate to heat up the skin of a subject at a given location based on the position of that sensor assembly 110 on garment 105. The co-located thermal sensing element 120 at that sensor assembly 110 may measure cooling of the subject's skin at that location. Heating elements 115 may thereby heat a subject's body at several locations, and then co-located thermal sensing elements 120 may measure a subject's skin's ability to cool (e.g., using thermistors) after turning off co-located heating elements 115. Heating element 115 may heat (e.g., heat up) the subject's skin at a given location without current flowing into the subject's body. Thermal spatial mapping of the cooling power on the subject's skin may be determined as further described below based on signals (e.g., sensed signals) provided to the exemplary disclosed system (e.g., controller 130) from sensor assemblies 110.

Turning back to FIG. 1, sensor assemblies 110 may be disposed in any desired location, pattern, configuration, and/or density on garment 105. For example, sensor assemblies 110 may be disposed in a grid pattern (e.g., a substantially evenly-spaced grid pattern) having any suitable spacing (e.g., between about 2 cm and about 12 cm, between about 2 cm and about 10 cm, between about 3 cm and about 8 cm, between about 4 cm and about 6 cm, or, for example, about 5 cm).

Sensor assemblies 110 may operate over any suitable time period or cycle. For example, heating elements 115 may heat skin of a subject wearing garment 105 for a few minutes (e.g., up to about 10 minutes, between about 3 minutes and about 10 minutes, and/or any other suitable time period). Heating elements 115 may be shut off and respective co-located thermal sensing elements 120 may measure temperature change (e.g., temperature decay) as a subject's skin cools. Thermal sensing elements 120 may measure temperature decay for any suitable time period such as, for example, up to about 10 minutes, up to about 5 minutes, between about 1 and about 10 minutes, between about 1 and 5 minutes, or any other suitable time period. Thermal sensing elements 120 of sensor assemblies 110 of garment 105 may simultaneously provide temperature signals. Some or all sensor assemblies 110 of garment 105 may operate simultaneously.

One or more connectors 125 may connect sensor assemblies 110 to one or more power sources and/or controllers (e.g., controller 130). Connector 125 may be any suitable type of electrical connector such as, for example, an electrical wire. Signals and power may be provided to sensor assemblies 110 via connector 125 (e.g., an electrical line, or an electrical cable). The heating elements 115 may be heated by receiving current from a set of first wires, and the set of first wires may be connected so as to send heating signals to the heating elements 115 individually and/or collectively. The thermal sensing elements 120 may provide signals indicative of temperatures sensed by the thermal sensing elements 120 over a set of second wires, and the set of second wires may be connected so as to receive individual signals from each thermal sensing element 120.

Controller 130 may control (e.g., send and receive signals) and power sensor assemblies 110 via connector 125. Controller 130 may control components (e.g., sensor assemblies 110) of garment 105 via connector 125 and/or wirelessly (e.g., via wireless communication, network communication, WiFi, Bluetooth, ZigBee, NFC, IrDA, and/or any other suitable communication technique). Controller 130 may provide power to sensor assemblies. Controller 130 may communicate with and receive signals and/or data from sensor assemblies 110. Controller 130 may be any suitable device for controlling, receiving, and/or processing signals and/or data of sensor assemblies 110. Controller 130 may be, for example, a computer apparatus as described below.

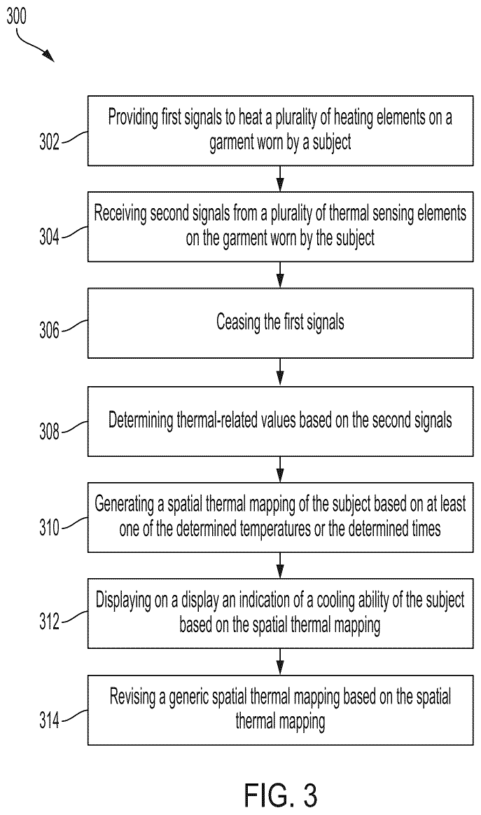

FIG. 3 illustrates an exemplary method for generating a spatial thermal mapping of a subject. Method 300 may be computer-implemented by one or more apparatuses or systems. While an order of operations is indicated in FIG. 3 for illustrative purposes, the timing and ordering of such operations may vary where appropriate without negating the purpose and advantages of the examples set forth in detail herein.

At step 302, the exemplary disclosed method may include providing first signals to heat a plurality of heating elements 115 on garment 105 worn by a subject. The first signals may be provided via any suitable communication technique, for example as described above. If the heating elements 115 are resistors, the first signals may be current provided to the resistors in an amount to heat the resistors. For example, the first signals may cause current to flow to the plurality of heating elements. The garment and associated wiring of the sensor assemblies 110 protect the subject from receiving any current driven to the heating elements, and as such, current does not flow to the subject when the first signals are provided.

At step 304, the exemplary disclosed method may include receiving second signals from a plurality of thermal sensing elements 120 on garment 105 worn by the subject. The second signals may be provided via any suitable communication technique, for example as described above. If the thermal sensing elements 120 are thermistors, the second signals may be signals to measure a resistance of each thermistor. The second signals may be received while the heating elements are heated and while the heating elements are cooled. The second signals may be received while the heating elements are cooled.

At step 306, the exemplary disclosed method may include ceasing the first signals. Ceasing the first signals may occur before (e.g., slightly before), simultaneously with, or after receiving the second signals. The first signals may be ceased after a certain amount of time or may be ceased after a certain temperature is achieved as measured by the second signals. The first signals may be ceased after the thermal sensing elements achieve at least a desired temperature, and a measured temperature of the thermal sensing elements to compare to the desired temperature may be based on the second signals. The first signals may be ceased after a first time period, where the first time period begins when the first signals are provided to the heating elements. For example, the first signals may be ceased after a few minutes (e.g., after about 10 minutes, after about 15 minutes, between about between about 1 minute and about 15minutes, between about 3 minutes and about 10 minutes, and/or any other suitable time period). For example, the certain temperature used to determine when to cease the first signals may be based on, for example, a minimum temperature for each sensor assembly 110 and/or an average temperature for all sensor assemblies 110.

After the first signals are ceased, the second signals may still be received for a second time period. The second time period may be any suitable time period such as, for example, up to about 10 minutes, up to about 5 minutes, between about 1 and about 10 minutes, between about 1 and 5 minutes, or any other suitable time period.

The second signals may be received during heating of the heating elements and during cooling of the heating elements. The second signals may be received during a cool down period of the heating elements, where the cool down period may, or may not, include a time for heating the heating elements.

At step 308, the exemplary disclosed method may include determining thermal-related values based on the second signals. For example, the thermal-related values may be determined by the controller 130 or by another computer apparatus. The thermal-related values may be at least one of: a temperature for each of the thermal sensing elements 120 after a cool down time period; an elapsed time for each of the thermal sensing elements 120 to cool down to a desired temperature; or a rate of temperature change for each of the thermal sensing elements 120 to cool down. The rate of temperature change for each of the thermal sensing elements may be determined based on a respective temperature and a respective time determined for each of the thermal sensing elements. A time to determine the rate of temperature change may be between about 1 minute to about 5 minutes, or between about 1 minute to about 10 minutes. The rate of temperature change for each of the thermal sensing elements may be a cooling rate and/or a heating rate determined based on the second signals.

At step 310, the exemplary disclosed method may include generating a spatial thermal mapping of the subject based on at least one of the determined thermal-related values from step 308, including the determined temperatures and the determined times. Generating the spatial thermal mapping may include: ranking the heating elements 115 (or the sensor assemblies 110) on the garment 105 based on the determined thermal-related values; and assigning a value on a normalized scale to each of the heating elements 115 (or the sensor assemblies 110) on the garment 105 based on ranking. Generating the spatial thermal mapping may include identifying heating elements 115 (or the sensor assemblies 110) on the garment 105 reaching a desired temperature during the second time period. Generating the spatial thermal mapping may include at least one of: identifying heating elements 115 (or the sensor assemblies 110) on the garment 105 decreasing to a desired temperature after ceasing the first signals; or identifying heating elements 115 (or the sensor assemblies 110) on the garment 105 not decreasing to a desired temperature after ceasing the first signals. Generating the spatial thermal mapping may include at least one of: identifying heating elements 115 (or the sensor assemblies 110) on the garment 105 decreasing to a desired temperature at a rate of temperature change above a first threshold; or identifying heating elements 115 (or the sensor assemblies 110) on the garment 105 decreasing to a desired temperature at a rate of temperature change below a second threshold. Generating the spatial thermal mapping may include at least one of: identifying heating elements 115 (or the sensor assemblies 110) on the garment 105 having a desired rate of temperature change above a first threshold; or identifying heating elements 115 (or the sensor assemblies 110) on the garment 105 having a desired rate of temperature change below a second threshold.

At step 312, the exemplary disclosed method may include displaying on a display an indication of a cooling ability of the subject based on the spatial thermal mapping determined at step 310. The display may be provided by the controller 130 or another computer apparatus. As an example, the cooling ability of the subject may be displayed with a representation of the subject and a representation of the spatial thermal mapping overlaid on the subject. The representation of the subject may be, for example, a user-rotatable three-dimensional view of the subject.

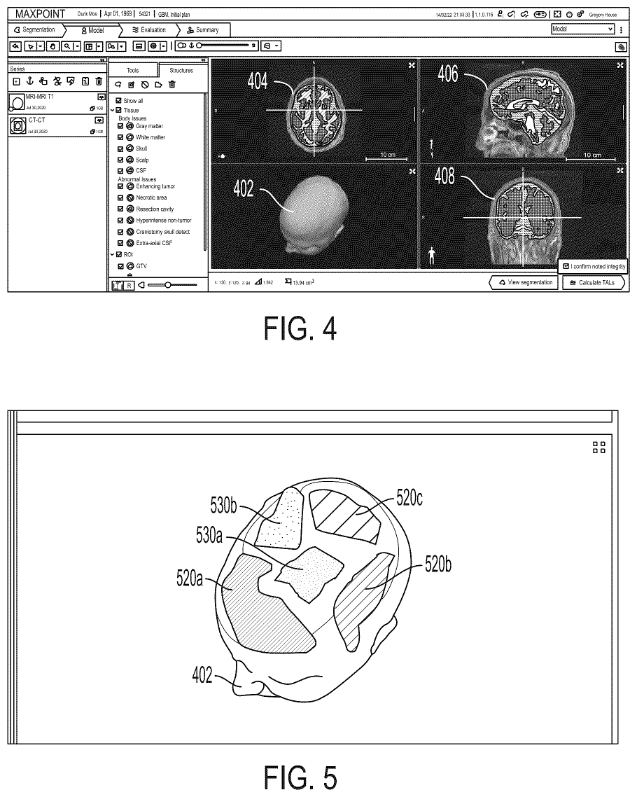

FIG. 4 illustrates an example display of a three-dimensional representation of a subject. The three-dimensional representation of the subject may include several user-selectable and user-rotatable views of the subject. For example, the three-dimensional representation of the subject may include a three-dimensional exterior view 402 of the subject, a first slice 404 in an axial plane through the subject, a second slice 406 in a sagittal plane through the subject, and a third slice 408 in a coronal plane through the subject. A user may select and/or rotate the exterior view 402, the first slice 404, the second slice 406, and the third slice 408 in unison or individually. The three-dimensional representation of the subject may be developed based imaging of the subject and used for treatment planning for applying TTFields to the subject.

FIG. 5 illustrates an example display of a subject based on a spatial thermal mapping of the subject. In particular, the example display provides an indication of a cooling ability of the subject based on the spatial thermal mapping of the subject. A three-dimensional representation of a subject may be the three-dimensional exterior view 402 of the subject having a representation of the spatial thermal mapping overlaid on the subject. The spatial thermal mapping may be represented as one or more locations on the subject that easily dissipate heat (or are easily cooled) and/or locations that do not easily dissipate heat (or are not easily cooled). For example, locations 520a, 520b, and 520c may indicate locations on the subject that easily dissipate heat (or are easily cooled), and locations 530a and 530b may indicate locations on the subject that do not easily dissipate heat (or are not easily cooled). For example, one color (e.g., blue) may be used to depict locations that easily dissipate heat, and another color (e.g., red) may be used to depict locations that do not easily dissipate heat. For example, color gradation and/or color opacity may be used to indicate magnitude of the ability to dissipate or not easily dissipate heat. For example, a ranking of the locations on the subject to dissipate or not easily dissipate heat (correlated to a ranking of the locations of the sensor assemblies) may be displayed using colors, color gradations, color opacities, and/or or patterns.

Returning to FIG. 3, at step 314, the exemplary disclosed method may include revising a generic spatial thermal mapping based on the spatial thermal mapping. For example, the generic spatial thermal mapping may be based on a database (e.g., a subject pool database) of a population of subjects including data of characteristics affecting treatment of the subjects. The generic spatial thermal mapping may be associated with a population group having in common at least one of, for example: gender, age, height, weight, body mass index, or race. The database of the population of subjects may be used to identify re-occurring patterns in population groups for spatial thermal mapping that may be used in TTFields treatment plans.

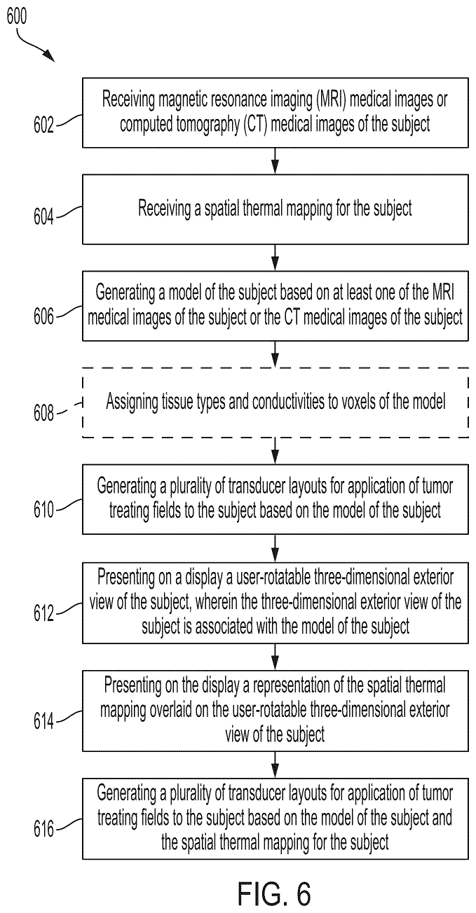

FIG. 6 illustrates an exemplary method for generating transducer layouts for applying TTFields to a subject based on a spatial thermal mapping of the subject. Method 600 may be computer-implemented by one or more apparatuses or systems. While an order of operations is indicated in FIG. 6 for illustrative purposes, the timing and ordering of such operations may vary where appropriate without negating the purpose and advantages of the examples set forth in detail herein.

At step 602, the exemplary disclosed method may include receiving at least one of magnetic resonance imaging (MRI) medical images or computed tomography (CT) medical images of the subject.

At step 604, the exemplary disclosed method may include receiving a spatial thermal mapping of the subject. The spatial thermal mapping of the subject may be received by accessing data representing the spatial thermal mapping stored in memory accessible by the computer and/or by receiving data representing the spatial thermal mapping over a network. In some embodiments, the spatial thermal mapping of the subject may be obtained as described above using method 300. In some embodiments, the spatial thermal mapping of the subject may be obtained using thermal imaging equipment. In some embodiments, the spatial thermal mapping of the subject may be obtained from a generic spatial thermal mapping developed from or selected from a database of subjects, such as the generic spatial thermal mapping of step 314 of FIG. 3.

At step 606, the exemplary disclosed method may include generating a model of the subject based on at least one of the MRI medical images of the subject or the CT medical images of the subject received at step 602. The model of the subject may include voxels. Generating the model of the subject may include generating a three-dimensional model of the subject. The three-dimensional model of the subject may be based on MRI medical images and CT medical images registered together. In particular, registration of medical images in different formats, such as the MRI medical images and CT medical images may include generating registered medical images, by registering image data in the first format to image data in the second format based on one or more of landmarks, transformation algorithm, a coordinate system of the object geometrical information, or dimensional information.

At step 608, the exemplary disclosed method may optionally include assigning tissue types and conductivities to voxels of the model. For example, the three-dimensional model may include voxels assigned tissue types and associated conductivities. The assigned tissue types may be assigned based on user input, automatically, or based on a combination of user input and automatically. The assigned conductivities may be assigned for each tissue type, and assigned conductivities may be assigned based on user input, automatically, or based on a combination of user input and automatically. In some embodiments, step 608 may not be performed, and as such, step 608 is depicted with a dashed outline in FIG. 6.

In some embodiments, instead of or in addition to assigning tissue types and conductivities to image voxels of the model, the exemplary disclosed method may include generating the model of the subject based on one or more geometrical relationships between a tumor and the subject.

At step 610, the exemplary disclosed method may include generating a plurality of transducer layouts for application of TTFields to the subject based on the model of the subject. In some embodiments, generating possible transducer layouts for application of TTFields to the subject may be based on computer simulations of the model of the subject to determine TTFields intensities. For example, using conductivities assigned at step 608, TTFields intensities of the model may be calculated for a variety of potential transducer locations on the subject and recommended locations may be identified which optimize TTFields intensity in specific areas of the subject. As an example, calculating TTFields intensities based on conductivities assigned at step 608 may involve generating simulation results for an induced electric field for a subject's body, for example, as discussed in U.S. Patent Application Publication No. 2022/0096829, which is incorporated herein by reference. As an example, generating simulation results may include: obtaining a three-dimensional model of AC electrical conductivity of a relevant anatomic volume; identifying the volume targeted for alternating electric field treatment within the three-dimensional model; automatically placing transducers on the three-dimensional model and setting relevant boundary conditions for the three-dimensional model; and calculating the electric field that develops within the model (e.g., using a finite element method analysis) once transducers have been placed on the model and boundary conditions applied. Recommended locations for placement of transducers on the subject may be identified to optimize TTFields intensity in specific areas of the subject.

In some embodiments, generating possible transducer layouts for application of TTFields to the subject may be based on the computer calculations using one or more geometrical relationships between a tumor and the subject. As an example, recommended transducer positions for applying TTFields may be determined using geometrical analysis, for example, as discussed in U.S. Patent Application Publication No. 2022/0148171, which is incorporated herein by reference. For example, a computer-implemented method may be used that includes geometric analysis based on a plurality of intersecting line segment pairs on an image of the subject, where each of the line segment pairs may correspond to locations to place transducers on the subject.

In some embodiments, generating possible transducer layouts for application of TTFields to the subject may be based on both computer simulations of the model of the subject to determine TTFields intensities and computer calculations using one or more geometrical relationships between a tumor and the subject.

In some embodiments, generating possible transducer layouts for application of TTFields to the subject may be based at least partially or entirely on the spatial thermal mapping of the subject.

At step 612, the exemplary disclosed method may include presenting on a display a user-rotatable three-dimensional exterior view of the subject, wherein the three-dimensional exterior view of the subject is associated with the model of the subject. The display may be provided similarly to as described above regarding step 312 in FIG. 3 and exemplary illustrated in FIG. 4.

At step 614, the exemplary disclosed method may include presenting on the display a representation of the spatial thermal mapping overlaid on the user-rotatable three-dimensional exterior view of the subject. Conductivity information may also be similarly displayed. The display may be provided similarly to as described above regarding step 314 in FIG. 3 and exemplary illustrated in FIG. 5.

At step 616, the exemplary disclosed method may include generating a plurality of transducer layouts for application of TTFields to the subject based on the model of the subject, the spatial thermal mapping for the subject, and/or the recommendation transducer positions determined at step 610. As an example, locations used in generating the transducer layouts may be weighted based on ability of locations on the subject to dissipate heat. The more heat that a given skin location on a subject may dissipate, the more current may be assigned to transducers at that location. For example, if a first point is able to dissipate 10% more heat than a second point, the first point may be assigned a weight of 1.1, and the second point may be assigned a weight of 1.0, which may quantify the additional capacity of the first point for dissipating heat as compared to the second point. Additionally, for example, a location on the subject having a low capability to dissipate heat could be assigned a weight less than 1.0 or significantly less than 1.0 (e.g., 0.55, which could be indicative of 50% of the heat dissipation ability of a location weighted at 1.1). For example, a location that cools relatively faster based on the spatial thermal mapping may be used as a location to place transducers for TTFields. For example, a location that cools relatively slowly based on the spatial thermal mapping may be avoided as a location to place transducers for TTFields. For example, current may be controlled during TTFields treatment based on whether certain locations cool relatively faster or slower than other locations (e.g., the system may assign more current to locations that cool faster). Further for example, a current provided to transducers may be varied based on the exemplary disclosed weighting described above. An increased amount of current may be directed to transducers at locations having a relatively higher weight for dissipating heat. A decreased amount of current may be directed to transducers at locations having a relatively lower weight for dissipating heat. Additionally, for example, an amount of current directed to transducers may be increased and/or decreased proportionally to the exemplary disclosed weighting of locations of the transducers to dissipate heat. Spatial thermal mapping may thereby be used as part of treatment planning for TTFields treatment.

In some embodiments, step 610 and step 616 may be combined.

In some embodiments, the spatial thermal mapping may be indicative (e.g., may also map) blood circulation and/or blood flow. For example, locations experiencing more cooling may be locations of more blood flow.

In some embodiments, garment 105 and an apparatus for applying TTFields (e.g., apparatus 700) may be separate components. In some exemplary embodiments, garment 105 and an apparatus for applying TTFields (e.g., apparatus 700) may be integrated together in a single component.

In some embodiments, an efficient and effective technique may be provided for measuring the ability of a subject's skin to cool. In some embodiments, a relatively inexpensive technique may be provided for spatial thermal mapping that may be implemented in a wide variety of subject locations and settings (e.g., in a medical facility or in a subject's residence). For example, an efficient and effective spatial thermal mapping with suitable precision and accuracy may be provided without involving cumbersome and expensive equipment and/or procedures.

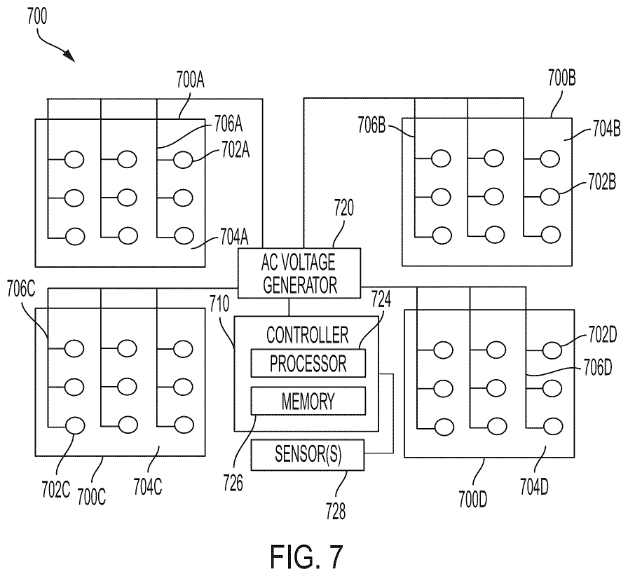

FIG. 7 illustrates a schematic view of an exemplary apparatus for applying alternating electric fields, such as TTFields. FIG. 7 depicts an example apparatus 700 to apply alternating electric fields (e.g., TTFields) to the subject's body. The system may be used for treating a target region of a subject's body with an alternating electric field. In an example, the target region may be in the subject's brain, and an alternating electric field may be delivered to the subject's body via two pairs of transducer arrays positioned on a head of the subject's body (such as, for example, in FIG. 9, which has four transducers 900). In another example, the target region may be in the subject's torso, and an alternating electric field may be delivered to the subject's body via two pairs of transducer arrays positioned on at least one of a thorax, an abdomen, or one or both thighs of the subject's body. Other transducer array placements on the subject's body may be possible.

The example apparatus 700 depicts an example system having four transducers (or “transducer arrays”) 700A-D. Each transducer 700A-D may include substantially flat electrode elements 702A-D positioned on a substrate 704A-D and electrically and physically connected (e.g., through conductive wiring 706A-D). The substrates 704A-D may include, for example, cloth, foam, flexible plastic, and/or conductive medical gel. Two transducers (e.g., 700A and 700D) may be a first pair of transducers configured to apply an alternating electric field to a target region of the subject's body. The other two transducers (e.g., 700B and 700C) may be a second pair of transducers configured to similarly apply an alternating electric field to the target region.

The transducers 700A-D may be coupled to an AC voltage generator 720, and the system may further include a controller 710 communicatively coupled to the AC voltage generator 720. The controller 710 may include a computer having one or more processors 724 and memory 726 accessible by the one or more processors. The memory 726 may store instructions that when executed by the one or more processors control the AC voltage generator 720 to induce alternating electric fields between pairs of the transducers 700A-D according to one or more voltage waveforms and/or cause the computer to perform one or more methods disclosed herein. The controller 710 may monitor operations performed by the AC voltage generator 720 (e.g., via the processor(s) 724). One or more sensor(s) 728 may be coupled to the controller 710 for providing measurement values or other information to the controller 710.

In some embodiments, the voltage generation components may supply the transducers 700A-D with an electrical signal having an alternating current waveform at frequencies in a range from about 50 kHz to about 1 MHz and appropriate to deliver TTFields treatment to the subject's body.

Electrode elements 702A-D may be capacitively coupled. In one example, electrode elements 702A-D are ceramic electrode elements coupled to each other via conductive wiring 706A-D. When viewed in a direction perpendicular to its face, the ceramic electrode elements may be circular shaped or non-circular shaped. In other embodiments, the array of electrode elements are not capacitively coupled, and there is no dielectric material (such as ceramic, or high dielectric polymer layer) associated with the electrode elements.

The structure of transducers 700A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements. Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer may include a polymer film that is adapted to generate an alternating electric field.

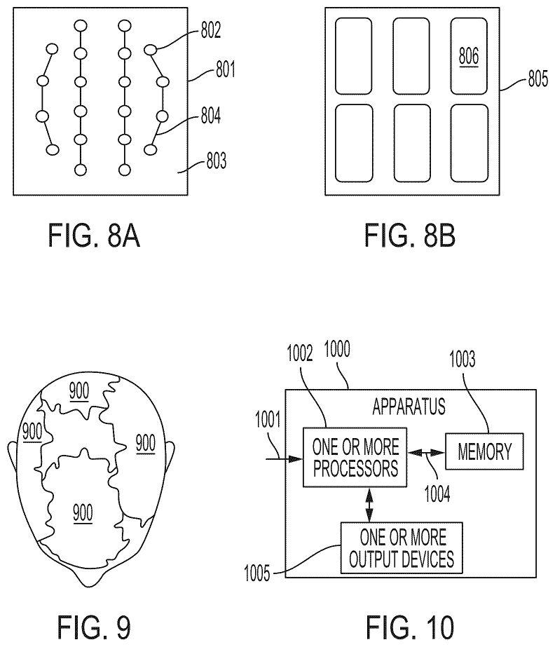

FIG. 8A illustrates a schematic view of an exemplary design of a transducer for applying alternating electric fields. Transducer 801 includes twenty electrode elements 802, which are positioned on substrate 803, and electrode elements 802 are electrically and physically connected to one another through a conductive wiring 804. In some embodiments, electrode elements 802 may include a ceramic disk.

FIG. 8B illustrates a schematic view of an exemplary design of a transducer for applying alternating electric fields. Transducer 805 may include substantially flat electrode elements 806. In some embodiments, electrode elements 806 are non-ceramic dielectric materials positioned over a plurality of flat conductors. Examples of non-ceramic dielectric materials positioned over flat conductors may include polymer films disposed over pads on a printed circuit board or over substantially planar pieces of metal. In some embodiments, such polymer films have a high dielectric constant, such as, for example, a dielectric constant greater than 10. In some embodiments, electrode elements 806 may have various shapes. For example, the electrode elements may be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 806 may have a same shape, similar shapes, and/or different shapes.

FIG. 10 depicts an example computer apparatus 1000 for use with the embodiments herein. As an example, apparatus 1000 may be a computer to implement certain inventive techniques disclosed herein, such as generating a spatial thermal mapping of a subject and/or selecting transducer locations for delivering TTFields to a subject based on the spatial thermal mapping of the subject. For example, method 300 of FIG. 3 may be performed by a computer, such as computer apparatus 1000. For example, method 600 of FIG. 6 may be performed by a computer, such as computer apparatus 1000, which may the same computer or a different computer than the computer used to perform method 300 of FIG. 3. In some embodiments, controller 130 of FIG. 1 may be implemented with apparatus 1000. In some embodiments, controller 710 to apply the alternating electric fields (e.g., TTFields) to a subject may be implemented with apparatus 1000. Apparatus 1000 may include one or more processors 1002, memory 1003, one or more input devices, and one or more output devices 1005 (e.g., a display or monitor).

In some embodiments, based on input 1001, the one or more processors 1002 may generate a spatial thermal mapping of a subject and/or select transducer locations for delivering TTFields to a subject based on the spatial thermal mapping of the subject as disclosed herein. As an example, input 1001 is user input. As an example, input 1001 may be from another computer in communication with apparatus 1000. Input 1001 may be received in conjunction with one or more input devices (not shown) of apparatus 1000.

Memory 1003 is accessible by one or more processors 1002 (e.g., via a link 1004) so that one or more processors 1002 can read information from and write information to memory 1003. Memory 1003 may store instructions that when executed by one or more processors 1002 implement one or more embodiments described herein. The memory 1003 may be a non-transitory computer readable medium (or a non-transitory processor readable medium) containing a set of instructions thereon for generating a spatial thermal mapping of a subject and/or selecting transducer locations for delivering TTFields to a subject based on the spatial thermal mapping of the subject, wherein when executed by a processor (such as one or more processors 1002), the instructions cause the processor to perform one or more methods disclosed herein.

One or more output devices 1005 may provide the status of the operation of the invention, such as displaying an indication of the spatial thermal mapping, transducer array selection, voltages being generated, and other operational information. Output device(s) 1005 may provide visualization data according to certain embodiments of the invention.

The apparatus 1000 may be an apparatus for generating a spatial thermal mapping of a subject and/or selecting transducer locations for delivering TTFields to a subject based on the spatial thermal mapping of the subject, the apparatus including: one or more processors (such as one or more processors 1002); and memory (such as memory 1003) accessible by the one or more processors, the memory storing instructions that when executed by the one or more processors, cause the apparatus to perform one or more methods disclosed herein.

### ILLUSTRATIVE EMBODIMENTS

The invention includes other illustrative embodiments (“Embodiments”) as follows.

Embodiment 1. A computer-implemented method for generating a spatial thermal mapping of a subject, the method comprising: providing first signals to heat a plurality of heating elements on a garment worn by the subject; receiving second signals from a plurality of thermal sensing elements on the garment worn by the subject; ceasing the first signals; determining thermal-related values based on the second signals, the thermal-related values comprising at least one of: a temperature for each of the plurality of thermal sensing elements after a cool down time period based on the second signals; a time needed for each of the plurality of thermal sensing elements to cool down to a desired temperature based on the second signals; or a rate of temperature change for each of the plurality of thermal sensing elements to cool down based on the second signals; and generating the spatial thermal mapping of the subject based on at least one of the determined temperatures or the determined times.

Embodiment 2: The computer-implemented method of Embodiment 1, wherein generating the spatial thermal mapping comprises: ranking the heating elements on the garment based on the determined thermal-related values; and assigning a value on a normalized scale to each of the heating elements on the garment based on ranking the heating elements.

Embodiment 2A: The computer-implemented method of Embodiment 1, wherein generating the spatial thermal mapping comprises: identifying heating elements on the garment reaching a desired temperature during a second time period.

Embodiment 2B: The computer-implemented method of Embodiment 1, wherein generating the spatial thermal mapping comprises at least one of: identifying heating elements on the garment decreasing to a desired temperature after ceasing the first signals; or identifying heating elements on the garment not decreasing to a desired temperature after ceasing the first signals.

Embodiment 2C: The computer-implemented method of Embodiment 1, wherein generating the spatial thermal mapping comprises at least one of: identifying heating elements on the garment decreasing to a desired temperature at a rate of temperature change above a first threshold; or identifying heating elements on the garment decreasing to a desired temperature at a rate of temperature change below a second threshold.

Embodiment 2D: The computer-implemented method of Embodiment 1, wherein generating the spatial thermal mapping comprises at least one of: identifying heating elements on the garment having a desired rate of temperature change above a first threshold; or identifying heating elements on the garment having a desired rate of temperature change below a second threshold.

Embodiment 3: The computer-implemented method of Embodiment 1, further comprising displaying on a display an indication of a cooling ability of the subject based on the spatial thermal mapping.

Embodiment 3A: The computer-implemented method of Embodiment 1, wherein the cooling ability of the subject may be displayed with a representation of the subject and a representation of the spatial thermal mapping overlaid on the subject.

Embodiment 4: The computer-implemented method of Embodiment 1, wherein the heating elements are resistors, and wherein the thermal sensing elements are thermistors.

Embodiment 4A: The computer-implemented method of Embodiment 1, wherein heating elements are co-located with thermal sensing elements on the garment.

Embodiment 5: The computer-implemented method of Embodiment 1, wherein the first signals cause current to flow to the plurality of heating elements.

Embodiment 5A: The computer-implemented method of Embodiment 1, wherein current does not flow to the subject when the first signals are provided.

Embodiment 5B: The computer-implemented method of Embodiment 1, wherein the first signals are ceased after the thermal sensing elements achieve at least a desired temperature.

Embodiment 5C: The computer-implemented method of Embodiment 5B, wherein a measured temperature of the thermal sensing elements to compare to the desired temperature is based on the second signals.

Embodiment 6: The computer-implemented method of Embodiment 5B, wherein the first signals are ceased after a first time period.

Embodiment 6A: The computer-implemented method of Embodiment 6, wherein the first time period begins when the first signals are provided and is between 1 minute and 15 minutes.

Embodiment 6B: The computer-implemented method of Embodiment 6, wherein the first time period begins when the first signals are provided and is between 3 minutes and 10 minutes.

Embodiment 7: The computer-implemented method of Embodiment 1, wherein the second signals are received during heating of the plurality of heating elements and during cooling of the plurality of heating elements.

Embodiment 7A: The computer-implemented method of Embodiment 1, wherein the cool down period includes a time for heating the plurality of heating elements.

Embodiment 7B: The computer-implemented method of Embodiment 1, wherein the cool down time period occurs after a time for heating the plurality of heating elements.

Embodiment 7C: The computer-implemented method of Embodiment 1, wherein the rate of temperature change for each of the plurality of thermal sensing elements is determined based on a respective temperature and a respective time determined for each of the plurality of thermal sensing elements.

Embodiment 7D: The computer-implemented method of Embodiment 7C, wherein the respective time to determine the cooling rate is between 1 minute to 5 minutes.

Embodiment 7E: The computer-implemented method of Embodiment 1, further comprising: determining a heating rate for each of the plurality of thermal sensing elements based on the second signals.

Embodiment 7F: The computer-implemented method of Embodiment 1, wherein the second signals are received for each of the thermal sensing elements.

Embodiment 7G: The computer-implemented method of Embodiment 1, wherein the cool down time period is between 1 minute and 10 minutes.

Embodiment 7H: The computer-implemented method of Embodiment 1, wherein the cool down time period is between 1 minute and 5 minutes.

Embodiment 8: The computer-implemented method of Embodiment 1, further comprising: revising a generic spatial thermal mapping based on the spatial thermal mapping.

Embodiment 8A: The computer-implemented method of Embodiment 1, wherein the generic spatial thermal mapping is associated with a population group having in common at least one of: gender, age, height, weight, body mass index, or race.

Embodiment 9. An apparatus for generating a spatial thermal mapping of a subject, the apparatus comprising: one or more processors; and memory accessible by the one or more processors, the memory storing instructions that when executed by the one or more processors, cause the apparatus to perform a method comprising: providing first signals to heat a plurality of heating elements on a garment worn by the subject; receiving second signals from a plurality of thermal sensing elements on the garment worn by the subject; ceasing the first signals; determining thermal-related values based on the second signals, the thermal-related values comprising at least one of: a temperature for each of the plurality of thermal sensing elements after a cool down time period based on the second signals; a time needed for each of the plurality of thermal sensing elements to cool down to a desired temperature based on the second signals; or a rate of temperature change for each of the plurality of thermal sensing elements to cool down based on the second signals; and generating a spatial thermal mapping of the subject based on at least one of the determined temperatures or the determined times.

Embodiment 10. A non-transitory processor readable medium containing a set of instructions thereon for generating a spatial thermal mapping of a subject, wherein when executed by a processor, the instructions cause the processor to perform a method comprising: providing first signals to heat a plurality of heating elements on a garment worn by the subject; receiving second signals from a plurality of thermal sensing elements on the garment worn by the subject; ceasing the first signals; determining thermal-related values based on the second signals, the thermal-related values comprising at least one of: a temperature for each of the plurality of thermal sensing elements after a cool down time period based on the second signals; a time needed for each of the plurality of thermal sensing elements to cool down to a desired temperature based on the second signals; or a rate of temperature change for each of the plurality of thermal sensing elements to cool down based on the second signals; and generating a spatial thermal mapping of the subject based on at least one of the determined temperatures or the determined times.

Embodiment 11. A garment for obtaining a spatial thermal mapping of a subject when worn by the subject, the garment comprising: a plurality of heating elements attached to the garment; a plurality of thermal sensing elements attached to the garment; a plurality of first wires to provide current to heat the plurality of heating elements; and a plurality of second wires to receive signals from the plurality of thermal sensing elements; wherein each heating element is co-located with a respective thermal sensing element, and wherein each thermal sensing element is located to measure a skin temperature of the subject when the garment is worn by the subject.

Embodiment 12: The garment of Embodiment 11, wherein the co-located heating elements and thermal sensing elements are arranged in a grid pattern on the garment.

Embodiment 12A: The garment of Embodiment 11, wherein a number of heating elements attached to the garment is between 5 and 100, wherein a number of thermal sensing elements attached to the garment is the same as the number of heating elements attached to the garment.

Embodiment 12B: The garment of Embodiment 11, wherein each heating element attached to the garment is spaced apart from a closest heating element attached to the garment by 2 cm to 12 cm.

Embodiment 12C: The garment of Embodiment 11, wherein each heating element attached to the garment is spaced apart from a closest heating element attached to the garment by 3 cm to 8 cm.

Embodiment 12D: The garment of Embodiment 11, wherein the heating elements are arranged in a grid pattern on the garment with a spacing of 3 cm to 8 cm between closest heating elements.

Embodiment 13: The garment of Embodiment 11, wherein the garment is a snug-fitting garment having a size and shape for wearing on at least one of a head, a torso, or a leg of the subject.

Embodiment 13A: The garment of Embodiment 11, wherein the garment is one of a vest, a helmet, a skull cap, or a legging.

Embodiment 14: The garment of Embodiment 11, wherein the heating elements are resistors, and wherein the thermal sensing elements are thermistors.

Embodiment 14A: The garment of Embodiment 11, wherein the heating elements are capable of being heated by receiving current from the plurality of first wires.

Embodiment 14B: The garment of Embodiment 11, wherein each heating element is located immediately adjacent to or within 0.5 cm of the respective thermal sensing element.

Embodiment 14C: The garment of Embodiment 11, wherein the thermal sensing elements are capable of providing signals indicative of temperatures sensed by the thermal sensing elements to the plurality of second wires.

Embodiment 14D: The garment of Embodiment 11, wherein the plurality of second wires are connected so as to receive individual signals from each thermal sensing element.

Embodiment 15. A computer-implemented method for generating at least one transducer layout for delivering tumor treating fields to a subject, the method comprising: receiving at least one of magnetic resonance imaging (MRI) medical images of the subject or computed tomography (CT) medical images of the subject; receiving a spatial thermal mapping of the subject; generating a model of the subject based on at least one of the MRI medical images of the subject or the CT medical images of the subject; and generating a plurality of transducer layouts for application of tumor treating fields to the subject based on the model of the subject and the spatial thermal mapping for the subject.

Embodiment 16: The computer-implemented method of Embodiment 15, wherein the spatial thermal mapping for the subject was obtained from thermal measurements of the subject.

Embodiment 16A: The computer-implemented method of Embodiment 16, wherein the thermal measurements of the subject were obtained from the subject wearing a garment for thermally measuring the subject.

Embodiment 16B: The computer-implemented method of Embodiment 16, wherein the thermal measurements of the subject were obtained from the subject using a thermal imaging camera.

Embodiment 16C: The computer-implemented method of Embodiment 15, wherein the spatial thermal mapping for the subject was selected from a database of spatial thermal mappings based on at least one of gender, age, height, weight, body mass index, or race of the subject. The database may be used to identify re-occurring patterns in population groups for spatial thermal mapping that may be used in treatment plans.

Embodiment 17: The computer-implemented method of Embodiment 15, further comprising: presenting on a display a user-rotatable three-dimensional exterior view of the subject, wherein the three-dimensional exterior view of the subject is associated with the three-dimensional model of the subject; and presenting on the display a representation of the spatial thermal mapping overlaid on the subject user-rotatable three-dimensional exterior view of the subject.

Embodiment 18: The computer-implemented method of Embodiment 15, wherein generating the model of the subject comprises: generating a three-dimensional model of the subject, wherein the three-dimensional model of the subject is based on MRI medical images and CT medical images registered together, wherein the three-dimensional model comprises voxels assigned tissue types and associated conductivities.

Embodiment 18A: The computer-implemented method of Embodiment 15, wherein the model of the subject is based on geometrically locating a tumor in the subject.

Embodiment 19. An apparatus for generating at least one transducer layout for delivering tumor treating fields to a subject, the apparatus comprising: one or more processors; and memory accessible by the one or more processors, the memory storing instructions that when executed by the one or more processors, cause the apparatus to perform a method comprising: receiving at least one of magnetic resonance imaging (MRI) medical images of the subject or computed tomography (CT) medical images of the subject; receiving a spatial thermal mapping of the subject; generating a model of the subject based on at least one of the MRI medical images of the subject or the CT medical images of the subject; and generating a plurality of transducer layouts for application of tumor treating fields to the subject based on the model of the subject and the spatial thermal mapping for the subject.

Embodiment 20. A non-transitory processor readable medium containing a set of instructions thereon for generating at least one transducer layout for delivering tumor treating fields to a subject, wherein when executed by a processor, the instructions cause the processor to perform a method comprising: receiving at least one of magnetic resonance imaging (MRI) medical images of the subject or computed tomography (CT) medical images of the subject; receiving a spatial thermal mapping of the subject; generating a model of the subject based on at least one of the MRI medical images of the subject or the CT medical images of the subject; and generating a plurality of transducer layouts for application of tumor treating fields to the subject based on the model of the subject and the spatial thermal mapping for the subject.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A computer-implemented method for generating a spatial thermal mapping of a subject, the method comprising:
providing first signals to heat a plurality of heating elements on a garment worn by the subject;
receiving second signals from a plurality of thermal sensing elements on the garment worn by the subject;
ceasing the first signals;
determining thermal-related values based on the second signals, the thermal-related values comprising at least one of:
a temperature for each of the plurality of thermal sensing elements after a cool down time period based on the second signals;
a time needed for each of the plurality of thermal sensing elements to cool down to a desired temperature based on the second signals; or
a rate of temperature change for each of the plurality of thermal sensing elements to cool down based on the second signals; and
generating the spatial thermal mapping of the subject based on at least one of the determined temperatures or the determined times.

2. The method of claim 1, wherein generating the spatial thermal mapping comprises:
ranking the heating elements on the garment based on the determined thermal-related values; and
assigning a value on a normalized scale to each of the heating elements on the garment based on ranking the heating elements.

3. The method of claim 1, wherein generating the spatial thermal mapping comprises at least one of:
identifying heating elements on the garment decreasing to a desired temperature after ceasing the first signals; or
identifying heating elements on the garment not decreasing to a desired temperature after ceasing the first signals.

4. The method of claim 1, wherein generating the spatial thermal mapping comprises at least one of:
identifying heating elements on the garment decreasing to a desired temperature at a rate of temperature change above a first threshold; or
identifying heating elements on the garment decreasing to a desired temperature at a rate of temperature change below a second threshold.

5. The method of claim 1, further comprising displaying on a display an indication of a cooling ability of the subject based on the spatial thermal mapping.

6. The method of claim 1, wherein the first signals are ceased after the thermal sensing elements achieve at least a desired temperature.

7. The method of claim 1, wherein the second signals are received during heating of the plurality of heating elements and during cooling of the plurality of heating elements.

8. The method of claim 1, wherein the rate of temperature change for each of the plurality of thermal sensing elements is determined based on a respective temperature and a respective time determined for each of the plurality of thermal sensing elements.

9. The method of claim 1, wherein the cool down time period is between 1 minute and 10 minutes.

10. The method of claim 1, further comprising:
revising a generic spatial thermal mapping based on the spatial thermal mapping.

11. A garment for obtaining a spatial thermal mapping of a subject when worn by the subject, the garment comprising:
a plurality of heating elements attached to the garment;
a plurality of thermal sensing elements attached to the garment;
a plurality of first wires to provide current to heat the plurality of heating elements; and
a plurality of second wires to receive signals from the plurality of thermal sensing elements;
wherein each heating element is co-located with a respective thermal sensing element,
wherein each thermal sensing element is located to measure a skin temperature of the subject when the garment is worn by the subject.

12. The garment of claim 11, wherein the co-located heating elements and thermal sensing elements are arranged in a grid pattern on the garment.

13. The garment of claim 11, wherein the garment is a snug-fitting garment having a size and shape for wearing on at least one of a head, a torso, or a leg of the subject.

14. The garment of claim 1, wherein the heating elements are resistors, and wherein the thermal sensing elements are thermistors.

15. The method of claim 1, wherein each heating element is located immediately adjacent to or within 0.5 cm of the respective thermal sensing element.

16. A computer-implemented method for generating at least one transducer layout for delivering tumor treating fields to a subject, the method comprising:
receiving at least one of magnetic resonance imaging (MRI) medical images of the subject or computed tomography (CT) medical images of the subject;
receiving a spatial thermal mapping of the subject;
generating a model of the subject based on at least one of the MRI medical images of the subject or the CT medical images of the subject; and
generating a plurality of transducer layouts for application of tumor treating fields to the subject based on the model of the subject and the spatial thermal mapping for the subject.

17. The method of claim 16, wherein the spatial thermal mapping for the subject was obtained from thermal measurements of the subject.

18. The method of claim 17, wherein the thermal measurements of the subject were obtained from the subject wearing a garment for thermally measuring the subject.

19. The method of claim 16, wherein the spatial thermal mapping for the subject was selected from a database of spatial thermal mappings based on at least one of gender, age, height, weight, body mass index, or race of the subject.

20. The method of claim 16, further comprising:
presenting on a display a user-rotatable three-dimensional exterior view of the subject, wherein the three-dimensional exterior view of the subject is associated with the model of the subject; and
presenting on the display a representation of the spatial thermal mapping overlaid on the subject user-rotatable three-dimensional exterior view of the subject.
